SEQUENCE LISTING (12 sequences)

SEQ ID NO: 1 - protein, 442 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 5 aa, Homo sapiens
EYYWS

SEQ ID NO: 3 - protein, 16 aa, Homo sapiens
EIFHSGSTNYNPSLKS

SEQ ID NO: 4 - protein, 8 aa, Homo sapiens
EELDDFDI

SEQ ID NO: 5 - protein, 116 aa, Homo sapiens
QVQLQQWGAGLLKPSETLSLTCAIYGGSFREYYWSWIRQPPGKGLEWIGEIFHSGSTNYNPSLKSRVTISVDTSKNQFSLKLSSVTAADTAVYYCAREELDDFDIWGQGTMVTVSS

SEQ ID NO: 6 - DNA, 1326 bp, Homo sapiens
  source 1..1326: mol_type = other DNA
caggtgcagctacagcagtggggcgcaggactgttgaagccttcggagaccctgtccctcacctgcgctatctatggtgggtccttcagggagtactactggagctggatccgccagcccccagggaaggggctggagtggattggggaaatctttcatagtggaagcaccaactacaacccgtccctcaagagtcgagtcaccatatcagtagacacgtccaagaaccagttctccctgaagctgagctctgtgaccgccgcggacacggctgtgtattactgtgcgagagaggaattagatgattttgatatctggggccaagggacaatggtcaccgtctcctcagcctccaccaagggcccatcggtcttccccctggcgccctgctccaggagcacctccgagagcacagcggccctgggctgcctggtcaaggactacttccccgaaccggtgacggtgtcgtggaactcaggcgctctgaccagcggcgtgcacaccttcccggctgtcctacagtcctcaggactctactccctcagcagcgtagtgaccgtgccctccagcaacttcggcacccagacctacacctgcaacgtagatcacaagcccagcaacaccaaggtggacaagacagttgagcgcaaatgttgtgtcgagtgcccaccgtgcccagcaccacctgtggcaggaccgtcagtcttcctcttccccccaaaacccaaggacaccctcatgatctcccggacccctgaggtcacgtgcgtggtggtggacgtgagccacgaagaccccgaggtccagttcaactggtacgtggacggcgtggaggtgcataatgccaagacaaagccacgggaggagcagttcaacagcacgttccgtgtggtcagcgtcctcaccgtcgtgcaccaggactggctgaacggcaaggagtacaagtgcaaggtctccaacaaaggcctcccagcccccatcgagaaaaccatctccaaaaccaaagggcagccccgagaaccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacacctcccatgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 7 - protein, 214 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 11 aa, Homo sapiens
RASQGISSWLA

SEQ ID NO: 9 - protein, 7 aa, Homo sapiens
AASSLQS

SEQ ID NO: 10 - protein, 9 aa, Homo sapiens
QQYKSYPRT

SEQ ID NO: 11 - protein, 107 aa, Homo sapiens [sequence omitted]

SEQ ID NO: 12 - DNA, 642 bp, Homo sapiens
  source 1..642: mol_type = other DNA
gacatccagatgacccagtctccatcctcactgtctgcatctgtaggagacagagtcaccatcacttgtcgggcgagtcagggtattagcagctggttagcctggtatcagcagaaaccagagaaagcccctaagtccctgatctatgctgcatccagtttgcaaagtggggtcccatcaaggttcagcggcagtggatctgggacagatttcactctcaccatcagtagcctgcagcctgaagattttgcaacttattactgccaacagtataaaagttaccctcggacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2025/0011416 A1
(43) Pub. Date: Jan. 9, 2025

(54) METHODS FOR THE TREATMENT OF CARDIOVASCULAR DISEASE

(71) Applicant: Tourmaline Bio, Inc., New York, NY (US)

(72) Inventors: Sandeep Kulkarni, New York, NY (US); Yung Chyung, New York, NY (US); Susan Dana Jones, New York, NY (US); Ryan Iarrobino, New York, NY (US); W. Bradford Middlekauff, New York, NY (US)

(21) Appl. No.: 18/888,316

(22) Filed: Sep. 18, 2024

Related U.S. Application Data
(63) Continuation of application No. PCT/US2023/078948, filed on Nov. 7, 2023.
(60) Provisional application No. 63/383,429, filed on Nov. 11, 2022.
(60) Provisional application No. 63/586,027, filed on Sep. 28, 2023.

(51) Int. Cl. C07K 16/24; A61K 39/00; A61P 9/00

(52) U.S. Cl. CPC C07K 16/248; A61P 9/00; A61K 2039/505; A61K 2039/54; A61K 2039/545

(57) ABSTRACT

The present disclosure provides methods of treating cardiovascular disease comprising subcutaneously administering to a patient in need thereof a therapeutically effective dose of an anti-interleukin-6 (anti-IL-6) antibody or antibody fragment. Further provided herein are pharmacologically active agents, compositions, methods and/or dosing schedules for the treatment of cardiovascular disease.

### CROSS-REFERENCE TO RELATED APPLICATIONS

This application claims priority to U.S. Provisional Patent Application No. 63/383,429, filed Nov. 11, 2022, and U.S. Provisional Patent Application No. 63/586,027, filed Sep. 28, 2023, each of which is incorporated by reference herein in its entirety for all purposes.

### REFERENCE TO AN ELECTRONIC SEQUENCE LISTING

The contents of the electronic sequence listing (TOUR_004_02WO_SeqList_ST26.xml; Size: 14,173 bytes; and Date of Creation: Nov. 3, 2023) are herein incorporated by reference in their entirety.

### TECHNICAL FIELD

The disclosure relates to therapeutic antibody molecules and treatments for cardiovascular disease.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke.

Interleukin-6 (IL-6) is a widely expressed cytokine that causes inflammation and oxidative stress, which would further result in cardiac injury. The increased expression of interleukin-6 is closely related to atherosclerosis, myocardial infarction, heart failure and ischemic stroke. Lipid lowering is the mainstay treatment for atherosclerosis. However, cardiovascular risk remains elevated even in patients with optimal lipid management.

IL-6 inhibition has been validated as a therapeutic strategy for reducing risk of cardiovascular (CV) morbidity and mortality. A phase II clinical trial of the human anti-IL-6 monoclonal antibody ziltivekimab shows that ziltivekimab substantially lowered various inflammatory biomarkers linked to atherosclerosis in advanced chronic kidney disease (CKD) patients (NCT03926117). However, anti-drug antibodies were detected in 12.5% of patients on 2-mg ziltivekimab, 6.3% of patients on 6-mg ziltivekimab and 12.5% of patients on 20-mg ziltivekimab.

Individuals with moderate chronic kidney disease and increases of IL-6 or C-reactive protein have markedly increased rates of major adverse cardiovascular events and comprise a group with substantial unmet needs for treatment.

### SUMMARY

Provided herein is a method of treating cardiovascular disease comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10.

In some embodiments, the anti-IL-6 antibody or antibody fragment comprises a heavy chain polypeptide comprising a polypeptide having at least about 98% identity to SEQ ID NO: 1 and a light chain polypeptide comprising a polypeptide having at least about 98% identity to SEQ ID NO: 7. In one aspect, the anti-IL-6 antibody or antibody fragment comprises a heavy chain polypeptide having the sequence of SEQ ID NO: 1 and a light chain polypeptide having the sequence of SEQ ID NO: 7.

In some embodiments, the patient being treated in accordance with methods of the disclosure is at least 18 years old.

In some embodiments, the patient being treated in accordance with methods of the disclosure has chronic kidney disease (CKD). In one embodiment, the patient has anemia of CKD. In another embodiment, the patient has Kidney Disease Outcomes Quality Initiative (KDOQI) stage 1-5 CKD. In another embodiment, the patient has Kidney Disease Outcomes Quality Initiative (KDOQI) stage 3-5 CKD. Still in another embodiment, the patient has below 60 mL/min/1.73 m\hat{ }2 in estimated glomerular filtration rate (eGFR) CKD-Epidemiology Collaboration (CKD-EPI).

In some embodiments, the patient being treated in accordance with methods of the disclosure has atherosclerotic cardiovascular disease (ASCVD), coronary artery disease (CAD) or peripheral artery disease (PAD).

In some embodiments, the patient has clonal hematopoiesis of indeterminate potential (CHIP). In some embodiments, the patient has one or more mutations in DNMT3A, TET2, ASXL1, PPMID, TP53, JAK2, SF3B1, or SRSF2.

In some embodiments, the patient has a risk factor for developing cardiovascular disease. For example and without limitation, the risk factor for developing cardiovascular disease is selected from the group consisting of (a) diabetes (Type I or Type II); (b) metabolic syndrome; (c) hypertension; (d) age about 55 or older for men or age about 65 or older for women; (c) dyslipidemia; (f) a family history of cardiovascular disease; (g) a history of stroke or transient ischemia attack; (h) smoking (currently active or prior history); (i) homocysteinemia; (j) hyperuricemia; (k) an HDL-C level of ≤about 40 mg/dL for men or ≤about 50 mg/dL for women; (l) renal dysfunction (e.g., a creatinine clearance (“CrCL”) of greater than about 30 mL/min and less than about 60 mL/min); (m) retinopathy (e.g., non-proliferative retinopathy, preproliferative retinopathy, proliferative retinopathy, maculopathy, advanced diabetic eye disease, or history of photocoagulation); (n) microalbuminuria (e.g., a positive microal or other strip test, an albumin/creatinine ratio of ≥about 2.5 mg/mmol, or an albumin excretion rate on timed collection of ≥about 20 mg/min all on at least two successive occasions); (o) macroalbuminuria (e.g., Albustix or other dip stick evidence of gross proteinuria, an albumin/creatinine ratio of ≥about 25 mg/mmol, or an albumin excretion rate on timed collection of ≥about 200 mg/min all on at least two successive occasions); and/or (p) an ankle-brachial index of <about 0.9 without symptoms of intermittent claudication.

In some embodiments, the patient being treated in accordance with methods of the disclosure has anemia of chronic inflammation, anemia of chronic disease, or iron-restricted anemia. In some embodiments, the patient has functional iron deficiency, or iron-restriction. In some embodiments, the patient has elevated serum or urinary hepcidin. In some embodiments, the patient has a red blood cell distribution width (RDW) of >about 13% or RDW in the highest quartile for the overall population. In some embodiments, the patient has a white blood cell count (WBC)>9000 cells per microliter of blood or WBC in the highest quartile for the overall population.

In some embodiments, the patient has recent infection within the past month, past 3 months, past 6 months, or past year. In some embodiments, the patient has recent COVID-19 (SARS-COV-2) infection within the past month, past 3 months, past 6 months, or past year. In some embodiments, the patient has recent surgery within the past month, past 3 months, past 6 months, or past year. In some embodiments, the patient has periodontal disease.

In some embodiments, the patient has an absolute neutrophil count of no less than 2.0×10⁹ per L. In some embodiments, the patient has a platelet count of no less than 120×10⁹ per L. In some embodiments, the patient has a spot urine to creatine ratio of no less than 4.

In some embodiments, the patient is negative for active tuberculosis, HIV, or hepatitis B or C. In some embodiments, the patient has not received a chronic use of immunosuppressive therapies.

In some embodiments, the cardiovascular disease is selected from a group consisting of nonfatal myocardial infarction, nonfatal stroke, and cardiovascular death. In one embodiment, the cardiovascular disease is heart failure.

In some embodiments, the therapeutically effective dose of the present disclosure is between about 5 mg to about 200 mg. In some embodiments, the therapeutically effective dose is about 5, about 7.5, about 10, about 15, about 20, about 25, about 30, about 50, about 60, about 70, about 80, about 90, or about 100 mg of the anti-IL-6 antibody or antibody fragment.

In some embodiments, the therapeutically effective dose is about 50 mg every 90 days. In one embodiment, the total therapeutically effective dose is about 100 mg. In some embodiments, the therapeutically effective dose is about 25 mg every 90 days. In one embodiment, the total therapeutically effective dose is about 50 mg. In some embodiments, the therapeutically effective dose is about 15 mg every 30 days. In one embodiment, the total therapeutically effective dose is about 90 mg.

In one embodiment, the therapeutically effective dose is administered subcutaneously.

In some embodiments, the dosing schedules for the anti-IL-6 antibody or antibody fragment is every 1 week to every 24 weeks. In one embodiment, the therapeutically effective dose is administered every 4, 8, 12 or 24 weeks. In some embodiments, the therapeutically effective dose is administered from every 30 days to every 90 days. In one embodiment, the therapeutically effective dose is administered every 30 days. In one embodiment, the therapeutically effective dose is administered every 90 days.

In some embodiments, the method of the present disclosure comprises: (a) administering a loading dose of the anti-IL-6 antibody or antibody fragment subcutaneously to the patient for at least the first two doses during a loading regimen; and (b) thereafter administering a maintenance dose of the anti-IL-6 antibody or antibody fragment subcutaneously to the patient during a maintenance regimen. In some embodiments, the loading regimen comprises administering the loading dose every 1 week, every 2 weeks, or every 4 weeks. In some embodiments, the maintenance regimen comprises administering the maintenance dose every 4 weeks, every 8 weeks, every 12 weeks, or every 24 weeks. In one embodiment, the method of the present disclosure comprises: (a) administering a loading dose of the anti-IL-6 antibody or antibody fragment subcutaneously to the patient every 4 weeks for the first two doses during a loading regimen; and (b) thereafter administering a maintenance dose of the anti-IL-6 antibody or antibody fragment subcutaneously to the patient every 8 or 12 weeks during a maintenance regimen. In some embodiments, the loading dose is greater than or equal to the maintenance dose. In some embodiments, the loading dose is between 5 mg to 200 mg. In some embodiments, the maintenance dose is between 5 mg to 200 mg.

In some embodiments, the patient has inflammation. In one embodiment, the patient has IL-6 mediated inflammation. In some embodiments, the treatment of the present disclosure is sufficient to reduce the inflammation without causing immune suppression.

In some embodiments, the immune suppression is measured by absolute neutrophil count (ANC). In some embodiments, the post-treatment ANC is at least 500 cells/pL. In some embodiments, the post-treatment ANC is at least 1000 cells/pL. In some embodiments, the post-treatment ANC is at least 1500 cells/pL. In some embodiments, the post-treatment ANC is at least 2000 cells/pL. In some embodiments, the ANC is decreased by no more than 2000 cells/pL as compared to pre-treatment levels. In some embodiments, the ANC is decreased by no more than 1500 cells/pL as compared to pre-treatment levels. In some embodiments, the ANC is decreased by no more than 1000 cells/pL as compared to pre-treatment levels. In some embodiments, the ANC is decreased by no more than 500 cells/pL as compared to pre-treatment levels. In some embodiments, the ANC is decreased by no more than about 50% as compared to pre-treatment levels. In some embodiments, the ANC is decreased by no more than about 40% as compared to pre-treatment levels. In some embodiments, the ANC is decreased by no more than about 30% as compared to pre-treatment levels. In some embodiments, the ANC is decreased by no more than about 20% as compared to pre-treatment levels. In some embodiments, the ANC is decreased by no more than about 10% as compared to pre-treatment levels. In some embodiments, the ANC is not decreased as compared to pre-treatment levels.

In some embodiments, inflammation is measured by high-sensitivity C-reactive protein (hsCRP) levels. In some embodiments, the patient has an elevated pre-treatment hsCRP level. In some embodiments, the pre-treatment hsCRP level of the patient is at least 2 mg/L. In some embodiments, the pre-treatment hsCRP level of the patient is at least 4 mg/L. In some embodiments, the pre-treatment hsCRP level of the patient is at least 6 mg/L. In some embodiments, the pre-treatment hsCRP level of the patient is at least 10 mg/L. In some embodiments, the pre-treatment hsCRP level of the patient is 2 mg/L or less. In some embodiments, the pre-treatment hsCRP level of the patient is 1 mg/L or less. In some embodiments, the post-treatment hsCRP level is no more than 2 mg/L. In some embodiments, the post-treatment hsCRP level is no more than 1 mg/L. In some embodiments, the hsCRP level is decreased by at least about 50% as compared to pre-treatment levels. In some embodiments, the hsCRP level is decreased by at least about 60% as compared to pre-treatment levels. In some embodiments, the hsCRP level is decreased by at least about 70% as compared to pre-treatment levels. In some embodiments, the hsCRP level is decreased by at least about 80% as compared to pre-treatment levels. In some embodiments, the hsCRP level is decreased by at least about 90% as compared to pre-treatment levels. In some embodiments, the treatment results in hsCRP reduction within about 4, about 8, about 12 or about 24 weeks of treatment. In some embodiments, the treatment results in hsCRP reduction after about 180 days of treatment. In some embodiments, the treatment is sufficient to sustain hsCRP reduction for at least 24, or 48 weeks.

In some embodiments, the treatment is sufficient to reduce fibrinogen, haptoglobin, serum amyloid A (SAA), secretory phospholipase A2 (sPLA2), lipoprotein (a), or neutrophil-to-lymphocyte ratio (NLR) by at least about 15%, about 30%, or about 50%.

In some embodiments, the treatment is sufficient to reduce the occurrence rate of one or more cardiovascular events by at least about 10%, about 15%, about 20%, about 25%, about 30%, about 40% or about 50%. In some embodiments, the adverse cardiovascular event is selected from the group consisting of death, cardiovascular death, non-fatal myocardial infarction, non-fatal stroke, unstable angina pectoris requiring urgent coronary revascularization, heart failure (new, worsening, or acute), ventricular arrhythmia due to ischemia, ischemic injury to cardiac valve, increase in N-terminal-pro-brain natriuretic peptide (NT-pro-BNP), an increase in cardiac marker of injury (such as troponin or creatine kinase-MB), a decrease in left ventricular ejection fraction of at least about 5%, atrial fibrillation or other supraventricular arrhythmia, bowel ischemia (small bowel or colon), new onset or worsening peripheral artery disease, critical limb ischemia, atheromatous embolism (cholesterol embolization), new onset or worsening thromboangiitis obliterans, worsening by one class or more in the New York Heart Association (NYHA) classification of heart failure, worsening score in the Kansas City Cardiomyopathy Questionnaire, and a change in short form 36 (SF-36) physical component score (PCS).

In some embodiments, the methods described herein further comprise a step of treating a subject with an additional form of therapy. In some embodiments, the additional form of therapy comprises administering one or more therapeutic agent in addition to the anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or a cardiovascular risk-modifying agent (e.g., anti-hypertensive drugs such as adrenergic blockers, angiotensin-converting enzyme inhibitors, angiotensin II receptor blockers, and calcium channel blockers; lipid lowering agents such as statins, fibrates, PCSK9 inhibitors, bile acid resins, niacin, selective cholesterol absorption inhibitors, omega-3 fatty acids and fatty acid esters, and adenosine triphosphate-citrate lyase (ACL) inhibitors; anti-diabetic drugs such as metformin; or anti-platelet agents such as aspirin, clopidogrel, ticlopidine, ticagrelor, prasugrel, and cangrelor).

In some embodiments, the anti-IL-6 antibody or antibody fragment containing said CDRs as described herein is contained in a pharmaceutical composition that comprises said anti-IL6 antibody or antibody fragment and a pharmaceutically acceptable carrier.

In some embodiments, provided are pharmacologically active agents, compositions, methods and/or dosing schedules that have certain advantages compared to the agents, compositions, methods and/or dosing schedules that are currently used and/or known in the art, including the ability to dose less frequently or to administer lower doses to obtain equivalent effects in inhibiting IL-6 mediated signaling.

### BRIEF DESCRIPTION OF THE DRAWINGS

FIG. 1 presents the schematic for the phase II randomized, double-blind, placebo-controlled trial of TOUR006.

### DETAILED DESCRIPTION

Provided herein are methods of treating cardiovascular disease comprising subcutaneously administering to a patient in need thereof a therapeutically effective dose of an anti-interleukin-6 (anti-IL-6) antibody or antibody fragment.

Further provided herein are pharmacologically active agents, compositions, methods and/or dosing schedules for the treatment of cardiovascular disease.

### Antibodies

Provided herein are antibodies and antigen-binding fragments thereof that specifically bind IL-6. Antibodies and antigen-binding fragments disclosed herein specifically bind human IL-6. In some embodiments, an antibody may be specific for only human IL-6 and may exhibit no non-human cross-reactivity.

Throughout the present disclosure, the term “about” may be used in conjunction with numerical values and/or ranges. The term “about” is understood to encompass values near to a recited value and within an acceptable degree of error in the art. For example, “about 40 [units]” may mean within ±10%, ±9%, ±8%, ±7%, ±6%, ±5%, ±4%, ±3%, ±2%, ±1%, less than ±1%, or any other value or range of values therein or there below.

As used herein, the term “antibody” refers to immunoglobulin (Ig) molecules and immunologically active portions or fragments of immunoglobulin molecules, i.e., molecules that contain an antigen-binding site that specifically binds (immunoreacts with) an antigen (e.g., IL-6). By “specifically binds” or “immunoreacts with” is meant that the antibody reacts with one or more antigenic determinants of the desired antigen and does not react with other polypeptides. In some embodiments, an antibody is said to specifically bind an antigen when it preferentially recognizes its target antigen in a complex mixture of proteins and/or macromolecules. In some embodiments, an antibody “specifically binds” IL-6 if the antibody binds IL-6 with greater affinity, greater avidity, more readily and/or for greater duration than it binds other polypeptides.

The term “antibody” broadly refers to an immunoglobulin (Ig) molecule, generally, comprising four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art.

In a full-length antibody, each heavy chain comprises a heavy chain variable domain (abbreviated herein as VH domain) and a heavy chain constant region. The heavy chain constant region comprises three domains, CH1, CH2 and CH3. Each light chain comprises a light chain variable domain (abbreviated herein as VL domain) and a light chain constant region. The light chain constant region comprises one domain, CL. The VH and VL domains can be further subdivided into regions of hypervariability, termed complementarity determining regions (CDRs), interspersed with regions that are more conserved, termed framework regions (FRs). Each VH domain and VL domain is composed of three CDRs and four FRs, arranged from amino-terminus to carboxyl-terminus in the following order: FR1, CDR1, FR2, CDR2, FR3, CDR3, FR4.

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is according to the EU numbering system. The Fc region of an immunoglobulin generally comprises two constant domains, CH2 and CH3. An Fc region can be present in dimer or monomeric form. The Fc region binds to various cell receptors, such as Fc receptors, and other immune molecules, such as complement proteins.

Immunoglobulin molecules can be of any type (e.g., IgG, IgE, IgM, IgD, IgA or IgY) and class (e.g., IgG1, IgG2, IgG3, IgG4, IgA1 or IgA2) or subclass. IgG, IgD, and IgE antibodies generally contain two identical heavy chains and two identical light chains and two antigen combining domains, each composed of a VH) and a VL. Generally IgA antibodies are composed of two monomers, each monomer composed of two heavy chains and two light chains (as for IgG, IgD, and IgE antibodies); in this way the IgA molecule has four antigen binding domains, each again composed of a VH and a VL. Certain IgA antibodies are monomeric in that they are composed of two heavy chains and two light chains. Secreted IgM antibodies are generally composed of five monomers, each monomer composed of two heavy chains and two light chains (as for IgG and IgE antibodies). Thus, the IgM molecule has ten antigen binding domains, each again composed of a VH and a VL. A cell surface form of IgM has a two heavy chain/two light chain structure similar to IgG, IgD and IgE antibodies.

The term “antigen-binding portion” or “antigen-binding fragment” of an antibody (or “antibody portion” or “antibody fragment”), as used herein, refers to one or more fragments of an antibody that retain the ability to specifically bind to an antigen (e.g., IL-6). It has been shown that the antigen-binding function of an antibody can be performed by portions or fragments of a full-length antibody. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CHI domains; (iv) a Fv fragment consisting of the VL and VH domains of a single arm of an antibody, (v) a dAb (domain antibody) fragment (Ward et al., (1989) Nature 341:544-546; WO 90/05144 A1, each herein incorporated by reference in its entirety), which comprises a single variable domain; and (vi) an isolated complementarity determining region (CDR). The disclosure also encompasses a Fab′ fragment. Fab′ fragments can be formed by the reduction of F(ab′)₂ fragments. Fab′ is derived from F(ab′)₂; therefore, it may contain a small portion of Fc. Furthermore, although the two domains of the Fv fragment, VL and VH, are coded for by separate genes, they can be joined, using recombinant methods, by a synthetic linker that enables them to be made as a single protein chain in which the VL and VH domains pair to form monovalent molecules (known as single chain Fv (scFv). See e.g., Bird et al. (1988) Science 242:423-426; Huston et al. (1988) Proc. Natl. Acad. Sci. USA 85:5879-5883. Such single chain antibodies are also intended to be encompassed within the term “antigen-binding portion” of an antibody. In some embodiments, scFv molecules may be incorporated into a fusion protein. In some embodiments, provided herein is a single chain camelid antibody. In some embodiments, provided herein is a shark heavy chain antibody (V-NAR). See, English et al. (2020) Antibody Therapeutics, 3(1):1-9. Examples of antigen-binding portions are known in the art (Kontermann and Dubel eds., Antibody Engineering (2001) Springer-Verlag. New York. 790 pp.). In some embodiments, provided herein is a single domain antibody. In general, the term “antibody” when used herein encompasses an “antibody fragment”. An antibody fragment generally retains the antigen-binding properties of a full-length antibody.

Antibodies and antibody portions provided herein may be in multispecific (e.g., bispecific or trispecific) formats. Such multispecific molecules specifically bind to two or more different molecular targets or epitopes. In some embodiments, an antibody or an antigen-binding portion is a bispecific molecule that binds specifically to a first antigen and a second antigen, wherein the first antigen is IL-6 and the second antigen is not IL-6. In some embodiments, an antibody or an antigen-binding portion is a diabody. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger et al. (1993) Proc. Natl. Acad. Sci. USA 90:6444-6448; Poljak et al. (1994) Structure 2:1121-1123). In some embodiments, an antibody or an antigen-binding portion is a triabody, a tetrabody, a bis-scFv or a tandem scFv. In some embodiments, an antibody or an antigen-binding portion is a dual affinity re-targeting protein.

In some embodiments, an anti-IL-6 antigen-binding portion disclosed herein is a Fab, a F(ab′)₂, a Fab′, a Fv, a scFv, a Fd, a single domain antibody, a single chain camelid antibody, a diabody, a triabody, a tetrabody or a bis-scFv.

As used herein, the terms “immunological binding” and “immunological binding properties” refer to the non-covalent interactions of the type which occur between an immunoglobulin molecule (e.g., antibody or antigen-binding portion thereof) and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See, Malmqvist, Nature 361:186-187 (1993)). The ratio of K_{off}/Kₒₙ enables the cancellation of all parameters not related to affinity, and is equal to the dissociation constant K_{d}. (See, Davies et al. (1990) Annual Rev Biochem 59:439-473). An antibody or antigen-binding portion provided herein is said to specifically bind IL-6 when the equilibrium binding constant (K_{d}) is ≤10 μM, preferably ≤10 nM, more preferably ≤10 nM, and most preferably ≤100 pM to about 1 pM, as measured by assays such as radioligand binding assays or similar assays known to those skilled in the art.

In some embodiments, an anti-IL-6 antibody or antigen-binding portion provided herein is monovalent or bivalent and comprises a single or double chain. Functionally, the binding affinity of an antibody or antigen-binding portion may be within the range of about 10⁻⁵M to 10⁻¹² M. For example, the binding affinity of an antibody or antigen-binding portion is from about 10⁻⁶ M to 10⁻¹² M, from about 10⁻⁷ M to 10⁻¹² M, from about 10⁻⁸ M to 10⁻¹² M, from about 10⁻⁹ M to 10⁻¹² M, from about 10⁻⁵ M to 10⁻¹¹ M, from about 10⁻⁶ M to 10⁻¹¹ M, from about 10⁻⁷ M to 10⁻¹¹ M, from about 10⁻⁸ M to 10⁻¹¹ M, from about 10⁻⁹ M to 10⁻¹¹ M, from about 10⁻¹⁰ M to 10⁻¹¹ M, from about 10⁻⁵ M to 10⁻¹⁰ M, from about 10⁻⁶ M to 10⁻¹⁰ M, from about 10⁻⁷ M to 10⁻¹⁰ M, from about 10⁻⁸ M to 10⁻¹⁰ M, from about 10⁻⁹ M to 10⁻¹⁰ M, from about 10⁻⁵ M to 10⁻⁹ M, from about 10⁻⁶ M to 10⁻⁹ M, from about 10⁻⁷ M to 10⁻⁹ M, from about 10⁻⁸ M to 10⁻⁹ M, from about 10⁻⁵ M to 10⁻⁸ M, from about 10⁻⁶ M to 10⁻⁸ M, from about 10⁻⁷ M to 10⁻⁸ M, from about 10⁻⁵ M to 10⁻⁷ M, from about 10⁻⁶ M to 10⁻⁷ M or from about 10⁻⁵ M to 10⁻⁶ M.

A human anti-IL-6 monoclonal antibody (PF-04236921) was described in U.S. Pat. No. 8,188,235, which is incorporated herein by reference in its entirety. The human anti-IL-6 monoclonal antibody is a fully human immunoglobulin G2 monoclonal antibody that binds to human IL-6 and has a half-life of 36⁻⁵¹ days. In phase I trials in healthy volunteers and patients with rheumatoid arthritis (protocol B0151001, NCT00838565 and NCT01166555), intravenous and subcutaneous (SC) the human anti-IL-6 monoclonal antibody (PF-04236921) was well tolerated and caused sustained suppression of C-reactive protein (CRP), a marker for inflammation that is transcriptionally controlled by IL-6. PF-04236921 has also been investigated in a phase II trial in patients with systemic lupus erythematosus (SLE; NCT01405196). While the study did not meet the primary end point, improvement was noted in the primary as well as key secondary end points with 10 mg. Overall, the human anti-IL-6 monoclonal antibody demonstrated desirable pharmacokinetic (PK) and pharmacodynamic (PD) properties supporting sustained target inhibition, and low incidence of immunogenicity upon single and multiple dose administration. See Danese, et al., Randomised trial and open-label extension study of an anti-interleukin-6 antibody in Crohn′s disease (ANDANTE I and II), Gut 2019;68:40-48; Li etal, Pharmacokinetics and C-reactive protein modelling of anti-interleukin-6 antibody (PF-04236921) in healthy volunteers and patients with autoimmune disease, Br J Clin Pharmacol. 2018 September; 84 (9): 2059-2074.

The amino acid and nucleic acid sequences of the human anti-IL-6 antibody (TOUR006) are provided in Table 1.

TABLE 1
Amino acid and nucleic acid sequences of human anti-IL-6 antibody

| Antibody name | Anti-IL-6 antibody (TOUR006) | |
|---|---|---|
| Domain or Region | Sequence | SEQ ID NO |
| Heavy chain | QVQLQQWGAGLLKPSETLSLTCAIYGGSFREYYW | 1 |
| (amino acid | SWIRQPPGKGLEWIGEIFHSGSTNYNPSLKSRVTIS | |
| sequence) | VDTSKNQFSLKLSSVTAADTAVYYCAREELDDFD | |
| | IWGQGTMVTVSSASTKGPSVFPLAPCSRSTSESTA | |
| | ALGCLVKDYFPEPVTVSWNSGALTSGVHTFPAVL | |
| | QSSGLYSLSSVVTVPSSNFGTQTYTCNVDHKPSNT | |
| | KVDKTVERKCCVECPPCPAPPVAGPSVFLFPPKPK | |
| | DTLMISRTPEVTCVVVDVSHEDPEVQFNWYVDG | |
| | VEVHNAKTKPREEQFNSTFRVVSVLTVVHQDWL | |
| | NGKEYKCKVSNKGLPAPIEKTISKTKGQPREPQVY | |
| | TLPPSREEMTKNQVSLTCLVKGFYPSDIAVEWESN | |
| | GQPENNYKTTPPMLDSDGSFFLYSKLTVDKSRWQ | |
| | QGNVFSCSVMHEALHNHYTQKSLSLSPGK | |
| Heavy chain CDR1 | EYYWS | 2 |
| Heavy chain CDR2 | EIFHSGSTNYNPSLKS | 3 |
| Heavy chain CDR3 | EELDDFDI | 4 |
| Heavy chain | QVQLQQWGAGLLKPSETLSLTCAIYGGSFREYYW | 5 |
| variable region | SWIRQPPGKGLEWIGEIFHSGSTNYNPSLKSRVTIS | |
| (VH) (amino acid | VDTSKNQFSLKLSSVTAADTAVYYCAREELDDFD | |
| sequence) | IWGQGTMVTVSS | |
| Heavy chain | caggtgcagctacagcagtggggcgcaggactgttgaagccttcggagacc | 6 |
| (nucleic acid | ctgtccctcacctgcgctatctatggtgggtccttcagggagtactactggagct | |
| sequence) | ggatccgccagcccccagggaaggggctggagtggattggggaaatctttca | |
| | tagtggaagcaccaactacaacccgtccctcaagagtcgagtcaccatatcag | |
| | tagacacgtccaagaaccagttctccctgaagctgagctctgtgaccgccgcg | |
| | gacacggctgtgtattactgtgcgagagaggaattagatgattttgatatctggg | |
| | gccaagggacaatggtcaccgtctcctcagcctccaccaagggcccatcggt | |
| | cttccccctggcgccctgctccaggagcacctccgagagcacagcggccctg | |
| | ggctgcctggtcaaggactacttccccgaaccggtgacggtgtcgtggaactc | |
| | aggcgctctgaccagcggcgtgcacaccttcccggctgtcctacagtcctcag | |
| | gactctactccctcagcagcgtagtgaccgtgccctccagcaacttcggcacc | |
| | cagacctacacctgcaacgtagatcacaagcccagcaacaccaaggtggac | |
| | aagacagttgagcgcaaatgttgtgtcgagtgcccaccgtgcccagcaccac | |
| | ctgtggcaggaccgtcagtcttcctcttccccccaaaacccaaggacaccctc | |
| | atgatctcccggacccctgaggtcacgtgcgtggtggtggacgtgagccacg | |
| | aagaccccgaggtccagttcaactggtacgtggacggcgtggaggtgcataa | |
| | tgccaagacaaagccacgggaggagcagttcaacagcacgttccgtgtggtc | |
| | agcgtcctcaccgtcgtgcaccaggactggctgaacggcaaggagtacaagt | |
| | gcaaggtctccaacaaaggcctcccagcccccatcgagaaaacc.atctcca | |
| | aaaccaaagggcagccccgagaaccacaggtgtacaccctgcccccatccc | |
| | gggaggagatgaccaagaaccaggtcagcctgacctgcctggtcaaaggctt | |
| | ctaccccagcgacatcgccgtggagtgggagagcaatgggcagccggaga | |
| | acaactacaagaccacacctcccatgctggactccgacggctccttcttcctct | |
| | acagcaagctcaccgtggacaagagcaggtggcagcaggggaacgtcttct | |
| | catgctccgtgatgcatgaggctctgcacaaccactacacgcagaagagcctc | |
| | tccctgtctccgggtaaa | |
| Light chain (amino | DIQMTQSPSSLSASVGDRVTITCRASQGISSWLAW | 7 |
| acid sequence) | YQQKPEKAPKSLIYAASSLQSGVPSRFSGSGSGTD | |
| | FTLTISSLQPEDFATYYCQQYKSYPRTFGQGTKVE | |
| | IKRTVAAPSVFIFPPSDEQLKSGTASVVCLLNNFYP | |
| | REAKVQWKVDNALQSGNSQESVTEQDSKDSTYS | |
| | LSSTLTLSKADYEKHKVYACEVTHQGLSSPVTKS | |
| | FNRGEC | |
| Light chain CDR1 | RASQGISSWLA | 8 |
| Light chain CDR2 | AASSLQS | 9 |
| Light chain CDR3 | QQYKSYPRT | 10 |
| Light chain variable | DIQMTQSPSSLSASVGDRVTITCRASQGISSWLAW | 11 |
| region (VL) (amino | YQQKPEKAPKSLIYAASSLQSGVPSRFSGSGSGTD | |
| acid sequence) | FTLTISSLQPEDFATYYCQQYKSYPRTFGQGTKVE | |
| | IK | |
| Light chain (nucleic | gacatccagatgacccagtctccatcctcactgtctgcatctgtaggagacaga | 12 |
| acid sequence) | gtcaccatcacttgtcgggcgagtcagggtattagcagctggttagcctggtat | |
| | cagcagaaaccagagaaagcccctaagtccctgatctatgctgcatccagttt | |
| | gcaaagtggggtcccatcaaggttcagcggcagtggatctgggacagatttca | |
| | ctctcaccatcagtagcctgcagcctgaagattttgcaacttattactgccaaca | |
| | gtataaaagttaccctcggacgttcggccaagggaccaaggtggaaatcaaa | |
| | cgaactgtggctgcaccatctgtcttcatcttcccgccatctgatgagcagttga | |
| | aatctggaactgcctctgttgtgtgcctgctgaataacttctatcccagagaggc | |
| | caaagtacagtggaaggtggataacgccctccaatcgggtaactcccaggag | |
| | agtgtcacagagcaggacagcaaggacagcacctacagcctcagcagcacc | |
| | ctgacgctgagcaaagcagactacgagaaacacaaagtctacgcctgcgaa | |
| | gtcacccatcagggcctgagctcgcccgtcacaaagagcttcaacagggga | |
| | gagtgt | |

CDR1, CDR2 and CDR3 (from left to right) sequences are underlined in the heavy chain and light
chain, respectively.

Provided herein is a method of treating cardiovascular disease comprising subcutaneously administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10. In some embodiments, said antibody or antibody fragment comprises a heavy chain polypeptide comprising a polypeptide having at least about 95%, about 96%, about 97%, about 98% or about 99% identity to SEQ ID NO: 1 and a light chain polypeptide comprising a polypeptide having at least about 95%, about 96%, about 97%, about 98% or about 99% identity to SEQ ID NO: 7. In some embodiments, said antibody or antibody fragment comprises a heavy chain polypeptide comprising a polypeptide having the sequence of SEQ ID NO: 1 and a light chain polypeptide comprising a polypeptide having the sequence of SEQ ID NO: 7. In some embodiments, the anti-IL-6 antibody or an antigen-binding portion comprises human IgG2 constant regions.

As used herein, the term “conservative substitution” refers to replacement of an amino acid with another amino acid which does not significantly deleteriously change the functional activity. A preferred example of a “conservative substitution” is the replacement of one amino acid with another amino acid which has a value≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | 1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4 |

Calculations of sequence homology or identity (the terms are used interchangeably herein) between sequences may be performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least about 30%, preferably at least about 40%, more preferably at least about 50%, even more preferably at least about 60%, and even more preferably at least about 70%, about 75%, about 80%, about 82%, about 84%, about 85%, about 86%, about 87%, about 88%, about 89%, about 90%, about 91%, about 92%, about 93%, about 94%, about 95%, about 96%, about 97%, about 98%, about 99%, or about 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, considering the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman et al. ((1970) J. Mol. Biol. 48:444-453) algorithm which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. In some embodiments, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) is a BLOSUM 62 scoring matrix with a gap penalty of 12, a gap extend penalty of 4, and a frameshift gap penalty of 5.

In some embodiments, the percent identity between two amino acid or nucleotide sequences can be determined using the algorithm of Meyers et al. ((1989) CABIOS 4:11-17) which has been incorporated into the ALIGN program (version 2.0), using a PAM120 weight residue table, a gap length penalty of 12 and a gap penalty of 4.

In some embodiments, the anti-IL-6 antibody or antigen-binding portion provided herein is monoclonal.

In some embodiments, the anti-IL-6 antibody or antigen-binding portion provided herein is chimeric. The term “chimeric” is intended to refer to an antibody molecule, or an antigen-binding portion thereof, in which the variable domain sequences are derived from one species and at least one constant region sequence is derived from another species. For example, one or all the variable domains of the light chain(s) and/or one or all the variable domains of the heavy chain(s) of a mouse antibody (e.g., a mouse monoclonal antibody) may each be joined to a human constant region, such as, without limitation an IgG1, IgG2, or an IgG4 human constant region. Examples of chimeric antibodies and suitable techniques for their generation are provided in U.S. Pat. Nos. 4,816,567; 4,975,369; and 4,816,397, each of which is incorporated herein by reference in its entirety.

In some embodiments, the anti-IL-6 antibody or antigen-binding portion provided herein is humanized. The term “humanized” is intended to refer to an antibody, or an antigen-binding portion thereof, that has been engineered to comprise one or more human framework regions in the variable domain together with non-human (e.g., mouse, rat, or hamster) CDRs of the heavy and/or light chain. In some embodiments, a humanized antibody comprises sequences that are entirely human except for the CDRs. In some embodiments, the VH domain, the VL domain, or both the VH domain and the VL domain of an anti-IL-6 antibody or antigen-binding portion provided herein comprise one or more human framework region amino acid sequences. In some embodiments, a humanized antibody comprises sequences that are entirely human except for the CDRs, which are the CDRs of antibody 32G8H6. Examples of humanized antibodies and suitable techniques for their generation are provided in Hwang et al., Methods 36:35, 2005; Queen et al., Proc. Natl. Acad. Sci. USA, 86:10029-10033, 1989; Jones et al., Nature, 321:522-25, 1986; Riechmann et al., Nature, 332:323-27, 1988; Verhoeyen et al., Science, 239:1534-36, 1988; Orlandi et al., Proc. Natl. Acad. Sci. USA, 86:3833-37, 1989; U.S. Pat. Nos. 5,225,539; 5,530,101; 5,585,089; 5,693,761; 5,693,762; 6,180,370; and WO 90/07861, each of which is incorporated herein by reference in its entirety.

In some embodiments, humanization comprises removal of post-translational modification (PTM) sites in the variable domain sequences (e.g., in the CDR or framework sequences) of a non-human antibody. For example, one or more PTM sites in CDR sequences may be removed by substituting certain amino acid residues. In some embodiments, humanization comprises CDR grafting and back mutation.

In some embodiments, the anti-IL-6 antibody or antigen-binding portion thereof comprises an immunoglobulin constant region. In some embodiments, the immunoglobulin constant region is IgG, IgE, IgM, IgD, IgA or IgY. In some embodiments, the immunoglobulin constant region is IgG1, IgG2, IgG3, IgG4, IgA1 or IgA2. In some embodiments, the immunoglobulin constant region is immunologically inert. In some embodiments, the immunoglobulin constant region comprises one or more mutations to reduce or prevent FcγR binding, antibody-dependent cell-mediated cytotoxicity activity, and/or complement-dependent cytotoxicity activity. In some embodiments, the immunoglobulin constant region is a wild-type human IgG1 constant region, a wild-type human IgG2 constant region, a wild-type human IgG4 constant region, a human IgG1 constant region comprising the amino acid substitutions L234A, L235A and G237A, a human IgG1 constant region comprising the amino acid substitutions L234A, L235A, G237A and P331S or a human IgG4 constant region comprising the amino acid substitution S228P, wherein numbering is according to the EU numbering system. In some embodiments, a position of an amino acid residue in a constant region of an immunoglobulin molecule is numbered according to EU nomenclature (Ward et al., 1995 Therap. Immunol. 2:77-94).

In some embodiments, the anti-IL-6 antibody or antigen-binding portion thereof may comprise an immunoglobulin light chain constant region that is a kappa light chain constant region or a lambda light chain constant region.

In some embodiments, the anti-IL-6 antibody or antigen-binding portion thereof may comprise a human IgG4 constant region comprising the amino acid substitution S228P and a kappa light chain constant region.

Further provided herein is an immunoconjugate comprising an anti-IL-6 antibody or an antigen-binding portion linked to a therapeutic agent. In some embodiments, the therapeutic agent is a small molecule drug.

### Pharmaceutical Compositions

The anti-IL-6 antibodies and antigen-binding portions described herein (also referred to herein as “active compounds”) can be incorporated into pharmaceutical compositions suitable for administration. Such compositions typically comprise an anti-IL-6 antibody or antigen-binding portion (or an immunoconjugate comprising said antibody or portion), and a pharmaceutically acceptable carrier, diluent or excipient. As used herein, the term “pharmaceutically acceptable” refers to molecular entities and compositions that do not generally produce allergic or other serious adverse reactions when administered using routes well known in the art. Molecular entities and compositions approved by a regulatory agency of the U.S. federal or state government or listed in the U.S. Pharmacopeia or other generally recognized pharmacopeia for use in animals, and more particularly in humans are considered to be “pharmaceutically acceptable.” As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington′s Pharmaceutical Sciences, a standard reference text in the field, which is incorporated herein by reference. Some examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

Provided herein is a pharmaceutical composition comprising (i) an anti-IL-6 antibody or an antigen-binding portion thereof, wherein the antibody or antigen-binding portion comprises a VH domain and a VL domain, wherein: (a) the VH domain amino acid sequence comprises HCDR1 of SEQ ID NO: 2, HCDR2 of SEQ ID NO: 3 and HCDR3 of SEQ ID NO: 4; and the VL domain amino acid sequence comprises LCDR1 of SEQ ID NO: 8, LCDR2 of SEQ ID NO: 9 and LCDR3 of SEQ ID NO: 10; and (ii) a pharmaceutically acceptable carrier, diluent or excipient.

A pharmaceutical composition disclosed herein may be formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primojel®, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical agents can also be prepared in the form of suppositories (e.g., with conventional suppository bases such as cocoa butter and other glycerides) or retention enemas for rectal delivery.

In some embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers.

It is especially advantageous to formulate oral or parenteral compositions in dosage unit form for case of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions provided herein can be included in a container, pack, or dispenser together with instructions for administration.

### Uses of Antibodies

Provided herein are methods and uses of the anti-IL-6 antibodies, anti-IL-6 antigen-binding portions, immunoconjugates and pharmaceutical compositions described herein for providing a therapeutic benefit to a subject with a condition associated with IL-6 expression. In some embodiments, the condition is an elevated cardiovascular risk. In some embodiments, the condition is chronic kidney disease (CKD) with an elevated cardiovascular risk. In some embodiments, the condition is cardiovascular disease including, but not limited to, nonfatal myocardial infarction, nonfatal stroke, cardiovascular death, and heart failure.

In some embodiments, the methods described herein further comprise a step of treating a subject with an additional form of therapy. In some embodiments, the additional form of therapy comprises administering one or more therapeutic agent in addition to the said anti-IL-6 antibody or antibody fragment as described herein. The therapeutic agents include, but are not limited to, a second antibody (e.g., an anti-IL-1 antibody, anti-IGF-1 receptor antibody, anti-VEGF antibody, and/or anti-IL17a antibody), a soluble receptor (e.g., soluble IL-1 receptor, soluble TNF-alpha receptor), an anti-inflammatory agent (e.g., paclitaxel, docetaxel, cisplatin, doxorubicin, prednisone, mitomycin, progesterone, tamoxifen, or fluorouracil), or a cardiovascular risk-modifying agent (e.g., anti-hypertensive drugs such as adrenergic blockers, angiotensin-converting enzyme inhibitors, angiotensin II receptor blockers, and calcium channel blockers; lipid lowering agents such as statins, fibrates, PCSK9 inhibitors, bile acid resins, niacin, selective cholesterol absorption inhibitors, omega-3 fatty acids and fatty acid esters, and adenosine triphosphate-citrate lyase (ACL) inhibitors; anti-diabetic drugs such as metformin; or anti-platelet agents such as aspirin, clopidogrel, ticlopidine, ticagrelor, prasugrel, and cangrelor).

Provided herein is an anti-IL-6 antibody or an anti-IL-6 antigen-binding portion, an immunoconjugate or a pharmaceutical composition described herein, for use as a medicament.

As used herein, the term “effective amount” or “therapeutically effective amount” refers to the amount of a pharmaceutical agent, e.g., an anti-IL-6 antibody or an antigen-binding portion thereof, which is sufficient to reduce or ameliorate the severity and/or duration of a disorder, e.g., cardiovascular disease, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). In some embodiments, the therapeutically effective dose of said anti-IL-6 antibody or antibody fragment is effective to change one or more biomarkers of IL-6 mediated signaling including, but not limited to, total sIL-6R, total IL-6, C-reactive protein (CRP), an/or autoantibody, for unexpectedly prolonged periods of time.

As used herein, the terms “treat,” “treating,” “treatment,” and the like refer to reducing or ameliorating a disorder, and/or signs or symptoms associated therewith, or slowing or halting the progression thereof. It will be appreciated that, although not precluded, treating a disorder or condition does not require that the disorder, condition or symptoms associated therewith be completely eliminated.

As used herein, “pre-treatment” means prior to the first administration of an anti-IL-6 antibody according the methods described herein. Pre-treatment does not exclude, and often includes, the prior administration of treatments other than an anti-IL-6 antibody.

As used herein, “post-treatment” means after the administration of an anti-IL-6 antibody according the methods described herein. Post-treatment includes after any administration of an anti-IL-6 antibody at any dosage described herein. Post-treatment also includes after the treatment phase of an anti-IL-6 antibody.

The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann J.A. et al., 1991, Int. J. Cancer 47:659-664; Bagshawe K.D. et al., 1991, Antibody, Immunoconjugates and Radiopharmaceuticals 4:915-922). Specific dosages may be indicated herein or in the Physician′s Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g., whole antibody, fragment) and the nature of any detectable label or other molecule attached to the antibody.

A typical antibody dose will be in the range 100 μg to 1 g for systemic applications, and 1 μg to 1 mg for intradermal injection. An initial higher loading dose, followed by one or more lower doses, may be administered. In some embodiments, the antibody is a whole antibody, e.g., the IgG1, IgG2 or IgG4 isotype. This is a dose for a single treatment of an adult subject, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for a subject may be dependent on the pharmacokinetic and pharmacodynamic properties of the antibody composition, the route of administration and the nature of the condition being treated. In some embodiments, the dosing of the present disclosure comprises an amount of at least about 10 mg, or at least about 20 mg, or at least about 30 mg, or at least about 40 mg, or at least about 50 mg of the anti-IL-6 antibody or antibody fragment.

Treatment may be periodic, and the period between administrations may be about two weeks or more, e.g., about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above. In some embodiments, the dosing schedules for the anti-IL-6 antibody or antibody fragment is once every 4 or 8 weeks up to about 52 total weeks.

In some embodiments, a subject is a human, a non-human primate, a pig, a horse, a cow, a dog, a cat, a guinea pig, a mouse or a rat. In some embodiments, a subject is an adult human. In some embodiments, a subject is a pediatric human. In some embodiments, the patient has previously received or is concurrently receiving prednisone or the like at the beginning of said treatment. In one aspect, the dosage of prednisone or the like is between 10 mg and 60 mg per day.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term′s definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

In the present description, any concentration range, percentage range, ratio range, or integer range is to be understood to include the value of any integer within the recited range and, when appropriate, fractions thereof (such as one tenth and one hundredth of an integer), unless otherwise indicated. It should be understood that the terms “a” and “an” as used herein refer to “one or more” of the enumerated components unless otherwise indicated. The use of the alternative (e.g., “or”) should be understood to mean either one, both, or any combination thereof of the alternatives. As used herein, the terms “include” and “comprise” are used synonymously.

The disclosure will be further clarified by the following examples, which are intended to be purely exemplary of the disclosure and in no way limiting.

### EXAMPLES

### Example 1: Clinical Evaluation of Human Anti-IL6 Antibody in Patients for Cardiovascular Disease

A Phase II clinical study of human anti-IL6 antibody TOUR006 as disclosed herein is prepared to conduct in patients at increased risk of cardiovascular events, including chronic kidney disease (CKD). The study is a randomized, double-blind, placebo-controlled trial designed to evaluate the anti-inflammatory effects of TOUR006 at three dosing regimens in participants with elevated cardiovascular risk (identified as hsCRP≥2.0 mg/L at baseline and a diagnosis of CKD).

### Inclusion Criteria

Participants must meet all of the following criteria to participate in the study:
1) Age≥18 years at time of Informed Consent Form (ICF) signature.
2) Serum hsCRP level≥2.0 mg/L at the Screening visit.
3) Diagnosis of CKD, (estimated glomerular filtration rate [eGFR]≥15 and <60 mL/min/1.73 m²) and not anticipated to require dialysis during the course of the study.
4) Received COVID-19 vaccine (completed the primary series) at least 30 days prior to the screening visit and/or had prior COVID-19 infection (except COVID-19 infection within 30 days prior to the Screening visit [see exclusion criterion #19]).
5) Agreement to comply with contraception and reproduction restrictions:
   a. Male participants must be surgically sterile or, if sexually active with a female partner of childbearing potential must also agree to use a condom for the duration of the study and 4 months after completion of study drug administration.
   b. Female participants of childbearing potential (including those with an onset of menopause <2 years before the Screening visit, non-therapy induced amenorrhea for <12 months before the Screening visit, or not surgically sterile [absence of ovaries and/or uterus and/or both fallopian tubes]) must have a negative serum pregnancy test at the Screening visit, negative urine pregnancy test at all protocol specified timepoints, and agree to use at least 1 acceptable method of contraception throughout the study and for 32 weeks after the last dose of study drug administration.

### Exclusion Criteria

Participants are excluded from the study if any of the following criteria apply:

### Laboratory Values

1) Hemoglobin values≤8.0 g/dL at the Screening visit.
2) Transferrin saturation<15.0% at the Screening visit.
3) Absolute neutrophil count<2.0×10⁹/L at the Screening visit.
4) Platelet count<120×10⁹/L at the Screening visit.
5) Spot urine protein to creatinine ratio>3000 mg/g (3.0 g/g) at the Screening visit.
6) Serum albumin<3.0 g/dL at the Screening visit.
7) Serum total IgG<700 mg/dL at the Screening visit.
8) Uncontrolled diabetes with HbA1c>9% at the Screening visit.
9) Fasting low density lipoprotein>130 mg/dL at the Screening visit.
10) Fasting triglycerides>200 mg/dL at the Screening visit.
11) Alanine aminotransferase (ALT) or aspartate aminotransferase (AST)>2.5×upper limit of normal at the Screening visit.
12) Positive for tuberculosis at the Screening visit. Blood testing (e.g., QuantiFERON) is preferred, but a purified protein derivative (PPD) skin test read within 48-72 hours by a qualified healthcare professional may also be performed. If a participant is positive by PPD but negative by QuantiFERON, the participant is eligible to enroll in the study.
13) Evidence of human immunodeficiency virus (HIV)-1 or HIV-2 infection by serology at the Screening visit.
14) Evidence of hepatitis B or C by serology (e.g., hepatitis B surface antigen or hepatitis C antibody positive) at the Screening visit.
15) Serum vitamin B-12 and folate levels below the laboratory lower limit of normal at the Screening visit (known or suspected causes of anemia other than CKD).

### Medical Conditions or Diseases

16) Acute coronary syndrome, ischemic stroke, transient ischemic attack, or other thrombotic or thromboembolic event within 6 months prior to randomization.
17) Participants with indwelling urinary or intravenous catheters.
18) Clinical evidence or suspicion of active infection.
19) History of major gastrointestinal bleeding (not including hemorrhoidal bleed) within 6 months prior to randomization.
20) History of active diverticulitis or gastrointestinal abscess within 12 months prior to randomization.
21) History of inflammatory bowel disease that has been clinically active within 12 months prior to randomization.
22) Uncontrolled hypertension (defined as an average of triplicate measurements systolic blood pressure>160 mmHg or an average diastolic blood pressure>100 mmHg prior to randomization). Participants may be reevaluated, at the discretion of the investigator, for this criterion if anti-hypertensive therapy has been started or increased as a result of initial screening blood pressure above these limits.
23) Planned coronary revascularization (percutaneous coronary intervention or coronary artery bypass grafting) or any other major surgical procedure during the course of the study.
24) History of major cardiac surgical, non-cardiac surgical, or major endoscopic procedure within 6 months prior to randomization.
25) New York Heart Association Class IV congestive heart failure.
26) History of hospitalization for heart failure exacerbation within past 3 months prior to randomization.
27) Untreated cardiac arrhythmia including:
   a. Hemodynamically significant arrhythmias
   b. Atrial fibrillation or atrial flutter with uncontrolled resting heart rate (>100 bpm)
   c. Any other arrhythmia that the investigator determines may impact the safety of the participant or confound the interpretation of safety data in this study.
28) QTcF>450 msec in males or 470 msec in females
29) Diagnosis of cachexia and/or unintended weight loss of >5% within 3 months prior to randomization.
30) Diagnosis of protein-losing enteropathy.
31) Actively treated or active malignancy within 12 months prior to randomization.
32) History of bone marrow or solid organ transplant or anticipated to receive a bone marrow or solid organ transplant during study participation.
33) Immunodeficiency (genetic or acquired, such as acquired immunodeficiency syndrome, common variable immunodeficiency, etc.).
34) Serious infection (an infection requiring hospitalization and/or intravenous [IV] antibiotic, IV antifungal, or IV antiviral treatment and/or having a clinical presentation that is viewed by the investigator as consistent with a serious infection) within the past 12 months before Screening or more than 1 such episode within 36 months prior to randomization.
35) COVID-19 infection (including mild or asymptomatic) that is ongoing or occurred within 30 days prior to randomization.
36) Any history (prior or current) of a serious opportunistic infection (e.g., not localized thrush due to corticosteroid therapy).
37) History of or clinically suspected systemic lupus erythematosus.
38) Any planned live attenuated vaccination during the course of this study.
39) Known allergy to the study drug or any of its ingredients.
40) Any history of substance use disorder, including alcohol abuse.
41) History of seizures within 10 years prior to randomization.
42) Any history of psychosis.
43) Significant cognitive impairment.

### Prior or Current Medications

44) Received an investigational drug within 30 days (or 5 half-lives of the investigational drug administered, whichever is longer) prior to randomization.
45) Any previous treatment with an immunomodulatory or immunosuppressive agent, including systemic corticosteroids, within 5 half-lives of the drug (or 3 months, whichever is longer) prior to randomization or anticipated use of such drugs any time during the study. Note: use of otic, ophthalmic, inhaled, and topical corticosteroids or local corticosteroid injections are not exclusionary.
46) Treatment with a hypoxia-inducible factor stabilizer (e.g., molidustat, Roxadustat) or an erythropoietin-mimetic (e.g., erythropoietin alpha or beta, darbopoietin alpha, or a continuous erythropoiesis receptor activator) within 6 weeks prior to randomization.
47) Expected to receive any investigational drug or any of the exclusionary drugs specified in the protocol during the Treatment Period or Safety Follow-Up Period.
48) Use of systemic antibiotics, antivirals, or antifungals (“systemic” is defined as oral or IV drugs that are absorbed into the circulation) within 14 days prior to randomization.

### General Exclusions

49) Currently breastfeeding.
50) Expected to need blood transfusions during the course of the study.
51) Any condition that could interfere with, or for which the treatment might interfere with, the conduct of the study or interpretation of the study results, or that would in the opinion of the investigator increase the risk of participating in the study.
52) Life expectancy anticipated to be <2 years.

Key inclusion criteria include chronic kidney disease (CKD), and pre-treatment hsCRP level of ≥2 mg/L. For example and without limitation, the eligible patients include those who have a pre-treatment hsCRP level of ≥2 mg/L, CKD, anemia of CKD, Kidney Disease Outcomes Quality Initiative (KDOQI) stage 1-5 CKD, below 60 mL/min/1.73 m\hat{ }2 in estimated glomerular filtration rate (eGFR) CKD-Epidemiology Collaboration (CKD-EPI), and/or have clonal hematopoiesis of indeterminate potential (CHIP) signatures. CHIP signatures include, but is not limited to, one or more mutations in DNMT3A, TET2, ASXL1, PPMID, TP53, JAK2, SF3B1, or SRSF2, found in cells of the blood or bone marrow.

The study also includes patients with established cardiovascular disease (CVD). For example and without limitation, eligible patients include those who have atherosclerotic cardiovascular disease (ASCVD), coronary artery disease (CAD) or peripheral artery disease (PAD).

Additionally, the study includes patients having a risk factor of developing cardiovascular disease regardless of having CKD or not. For example and without limitation, the risk factor for developing cardiovascular disease is selected from the group consisting of (a) diabetes (Type I or Type II); (b) metabolic syndrome; (c) hypertension; (d) age about 55 or older for men or age about 65 or older for women; (e) dyslipidemia; (f) a family history of cardiovascular disease; (g) a history of stroke or transient ischemia attack; (h) smoking (currently active or prior history); (i) homocysteinemia; (j) hyperuricemia; (k) an HDL-C level of ≤about 40 mg/dL for men or ≤about 50 mg/dL for women; (l) renal dysfunction (e.g., a creatinine clearance (“CrCL”) of greater than about 30 mL/min and less than about 60 mL/min); (m) retinopathy (e.g., non-proliferative retinopathy, preproliferative retinopathy, proliferative retinopathy, maculopathy, advanced diabetic eye disease, or history of photocoagulation); (n) microalbuminuria (e.g., a positive microal or other strip test, an albumin/creatinine ratio of ≥about 2.5 mg/mmol, or an albumin excretion rate on timed collection of ≥about 20 mg/min all on at least two successive occasions); (o) macroalbuminuria (e.g., Albustix or other dip stick evidence of gross proteinuria, an albumin/creatinine ratio of ≥about 25 mg/mmol, or an albumin excretion rate on timed collection of ≥about 200 mg/min all on at least two successive occasions); and (p) an ankle-brachial index of <about 0.9 without symptoms of intermittent claudication.

The study may also include patients who have anemia of chronic inflammation, anemia of chronic disease, or iron-restricted anemia; have functional iron deficiency, or iron-restriction; have elevated hepcidin (serum or urine); have a red blood cell distribution width (RDW) of about >13% or RDW in the highest quartile for the overall population; and/or have white blood cell count (WBC)>9000 cells per microliter of blood or WBC in the highest quartile for the overall population.

The study may also include patients who have recent infection within the past month, past 3 months, past 6 months, or past year; have recent COVID-19 (SARS-COV-2) infection within the past month, past 3 months, past 6 months, or past year; have recent surgery within the past month, past 3 months, past 6 months, or past year; and/or have periodontal disease.

The study may also include patients who have an absolute neutrophil count of no less than 2.0×10⁹ per L; have a platelet count of no less than 120×10⁹ per L; and have a spot urine to creatine ratio of no less than 4.

The study excludes those what are tested positive for active tuberculosis, HIV, or hepatitis B or C. The study also excludes those who have received a chronic use of immunosuppressive therapies.

### Dose Regimens

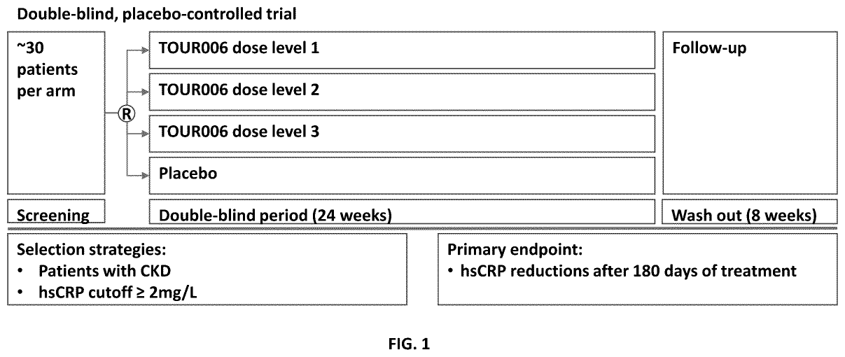

The study schematic is shown in FIG. 1. The eligible patents will be randomized 1:1:1:1 to 1 of 3 dosing regimens of TOUR006 or placebo (see treatment arms below). To mitigate against imbalances in background safety risk from CKD, participants will be stratified by CKD stage to ensure balanced randomization. The treatment arms are as follows:
Treatment Arm A (TOUR006 50 mg administered subcutaneously every 90 days)
   Participants will receive TOUR006 50 mg injections on Study Days 1 and 90; matching placebo injections will be administered on Study Days 30, 60, 120, and 150.
   Total cumulative TOUR006 dose: 100 mg
Treatment Arm B (TOUR006 25 mg administered subcutaneously every 90 days)
   Participants will receive TOUR006 25 mg injections on Study Days 1 and 90; matching placebo injections will be administered on Study Days 30, 60, 120, and 150.
   Total cumulative TOUR006 dose: 50 mg
Treatment Arm C (TOUR006 15 mg administered subcutaneously every 30 days)
   Participants will receive TOUR006 15 mg injections on Study Days 1, 30, 60, 90, 120, and 150.
   Total cumulative TOUR006 dose: 90 mg
Placebo: administered subcutaneously every 30 days

Approximately 120 participants (30 per treatment arm) will be randomized. After the screening period, randomized participants will be dosed every 30 days with TOUR006 or placebo (participants in Arms A and B will receive placebo at Study Days 30, 60, 120, and 150 to align with the 30-day dosing regimen). Participants will be followed for pharmacokinetic and pharmacodynamic endpoints through Study Day 180 and followed for safety until Study Day 365. The primary pharmacodynamic endpoint evaluation will occur at Study Day 180.

Additional TOUR006 dose regimens would include three dose levels in the range of 5 mg to 200 mg. For example, the effective dose would be 5, 7.5, 10, 15, 20, 25, 30 or 50 mg of the TOUR006 antibody. TOUR006 would be administered subcutaneously every 4, 8, 12, or 24 weeks. The total study duration for an individual patient is approximately 24 weeks.

Further dose regimens comprise: (a) administering a loading dose of the TOUR006 subcutaneously to the patient for the at least first two doses during a loading regimen; and (b) thereafter administering a maintenance dose of the TOUR006 subcutaneously to the patient during a maintenance regimen. The loading regimen comprises administering the loading dose every 1 week, every 2 weeks, or every 4 weeks. The maintenance regimen comprises administering the maintenance dose every 4 weeks, every 8 weeks, every 12 weeks, or every 24 weeks. The loading dose may be greater than or equal to the maintenance dose. The loading dose is between 5 mg to 200 mg and the maintenance dose is between 5 mg to 200 mg.

### Endpoints

Clinically relevant parameters including, but not limited to, high-sensitivity C-reactive protein (hsCRP), absolute neutrophil count (ANC), fibrinogen, haptoglobin, serum amyloid A (SAA), secretory phospholipase A2 (sPLA2), lipoprotein (a) level, neutrophil-to-lymphocyte ratio (NLR), red blood cell distribution width (RDW), LDL level, APOB, APOA1, hemoglobin, transferrin saturation (TSAT), reticulocyte hemoglobin count (Chr), total iron binding capacity (TIBC), serum ferritin, albumin, erythropoietic resistive index (ERI), handgrip, NT-proBNP, and cardiac MRI would be recorded during the study. Details of these clinically relevant parameters are described in U.S. Pat. Nos. 8,906,964, 11,369,582, and 11,384,143, the contents of each of which are hereby expressly incorporated by reference in their entirety for any purpose.

The primary endpoint of this study would be change from baseline in hsCRP after 180 days of treatment. Additionally, the primary endpoint would be a reduction of hsCRP from week 12 to week 24 of the treatment, for example, at week 12, week 16, or week 24 of the treatment. The enrolled patients would have inflammation or IL6-mediated inflammation. C-reactive protein (CRP) is a marker of inflammation. CRP levels increase in response to inflammation, and can be measured with an hsCRP (high-sensitivity C-reactive protein) test. The pre-treatment hsCRP of the patients is typically greater than 2 mg/L. Under certain circumstances, the pre-treatment hsCRP level of the patient is 1 mg/L or less. The treatment would be sufficient to reduce the hsCRP level. For example, the post-treatment hsCRP level is no more than 2 mg/L or 1 mg/L. The treatment would be sufficient to reduce the hsCRP level by at least about 50%, about 60%, about 70%, about 80%, or about 90% as compared to pre-treatment levels (baseline). The treatment would result in hsCRP reduction within 4, 8, 12, 16 or 24 weeks of treatment. Further, the treatment would be sufficient to sustain hsCRP reduction for at least 24, or 48 weeks.

One secondary endpoint is proportion of participants achieving hsCRP response (>90% decrease from baseline and/or hsCRP<2.0 mg/L) after 180 days of treatment. The other secondary endpoint is serum drug concentrations of TOUR006 at baseline and after 30, 60, 90, 120, 150, and 180 days of treatment and serum drug concentrations of TOUR006 at 210, 270, 330, and 365 days.

To evaluate the safety and tolerability of TOUR006 in participants with elevated cardiovascular risk and CKD, the safety endpoints include proportion of participants with adverse events (AEs), serious AEs (SAEs), severe AEs, and AEs leading to discontinuation; description and frequency of events of special interest by treatment group; and description of additional safety assessments by treatment group and dose, e.g., vital signs, electrocardiogram, and anti-drug antibodies.

To explore the effects of TOUR006 on markers of inflammation, the exploratory endpoints include change from baseline in serum amyloid A (SAA); change from baseline in lipoprotein (a); change from baseline in neutrophil-to-lymphocyte ratio (NLR); and change from baseline in secretory phospholipase (sPLA2). To evaluate the effects of TOUR006 on functional iron deficiency and anemia, additional endpoints include change from baseline in red blood cell distribution width (RDW); change from baseline in hemoglobin; and change from baseline in transferrin saturation (TSAT), reticulocyte hemoglobin count (Chr), total iron binding capacity (TIBC) and serum ferritin.

The treatment would be sufficient to reduce the inflammation without causing immune suppression. The immune suppression is measured by absolute neutrophil count (ANC). The normal range of ANC is about 500 to 7000 cells/pL; the post-treatment ANC would be at least 500, 1000, 1500, or 2000 cells/pL as compared to pre-treatment levels. Under certain circumstances, the ANC is decreased by no more than about 50%, about 40%, about 30%, about 20% or about 10% as compared to pre-treatment levels. Under certain circumstances, the ANC is not decreased as compared to pre-treatment levels.

Other secondary endpoints of this study include the occurrence rate of one or more cardiovascular events. The treatment would reduce the occurrence rate of one or more cardiovascular events by at least about 10%, about 15%, about 20%, about 25%, about 30%, about 40% or about 50%. The adverse cardiovascular events include, but is not limited to, death, cardiovascular death, non-fatal myocardial infarction, non-fatal stroke, unstable angina pectoris requiring urgent coronary revascularization, heart failure (new, worsening, or acute), ventricular arrhythmia due to ischemia, ischemic injury to cardiac valve, increase in N-terminal-pro-brain natriuretic peptide (NT-pro-BNP), an increase in cardiac marker of injury (such as troponin or creatine kinase-MB), a decrease in left ventricular ejection fraction of at least about 5%, atrial fibrillation or other supraventricular arrhythmia, bowel ischemia (small bowel or colon), new onset or worsening peripheral artery disease, critical limb ischemia, atheromatous embolism (cholesterol embolization), new onset or worsening thromboangiitis obliterans, worsening by one class or more in the New York Heart Association (NYHA) classification of heart failure, worsening score in the Kansas City Cardiomyopathy Questionnaire, and a change in short form 36 (SF-36) physical component score (PCS).

The study consists of three phases: (i) screening and randomization, (ii) treatment, and (iii) follow-up. During the screening, each potential subject will provide informed consent before starting any study-specific procedures. The randomization of subjects to study groups will be performed centrally by an Interactive Web-Response System (IWRS) using a randomization scheme reviewed and approved by an independent statistician. During the treatment period, randomized subjects will be provided the treatment and assessment according to the protocol. Follow-up will occur 8 weeks, 12 weeks, 16 weeks, 24 weeks, or 36 weeks following termination of the treatment.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1-64. (canceled)

65. A method of treating cardiovascular disease comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10.

66. The method of claim 65, wherein the antibody or antibody fragment comprises a heavy chain polypeptide comprising a polypeptide having at least 95% identity to SEQ ID NO: 1 and a light chain polypeptide comprising a polypeptide having at least 95% identity to SEQ ID NO: 7.

67. The method of claim 65, wherein the antibody or antibody fragment comprises a heavy chain polypeptide having the sequence of SEQ ID NO: 1 and a light chain polypeptide having the sequence of SEQ ID NO: 7.

68. The method of claim 65, wherein the therapeutically effective dose is between about 5 mg to about 200 mg.

69. The method of claim 68, wherein the therapeutically effective dose is about 5, about 7.5, about 10, about 15, about 20, about 25, about 30, about 40, about 50, about 60, about 70, about 80, about 90, or about 100 mg.

70. The method of claim 65, wherein the patient has chronic kidney disease (CKD).

71. A method of treating cardiovascular disease comprising administering to a patient in need thereof a therapeutically effective dose of an anti-interleukin-6 (anti-IL-6) antibody or antibody fragment having the variable heavy (VH) CDRs as defined in SEQ ID NOs 2, 3 and 4, and the variable light (VL) CDRs as defined in SEQ ID NOs 8, 9 and 10, wherein the therapeutically effective dose is about 25 mg, 30 mg, 40 mg, or 50 mg every 90 days, or wherein therapeutically effective dose is about 15 mg every 30 days.

72. The method of claim 71, wherein the therapeutically effective dose is administered subcutaneously.

73. The method of claim 71, wherein the patient is at least 18 years old.

74. The method of claim 71, wherein the patient has CKD.

75. The method of claim 65, wherein the patient has inflammation.

76. The method of claim 75, wherein the inflammation is measured by high-sensitivity C-reactive protein (hsCRP) levels.

77. The method of claim 76, wherein the patient has a pre-treatment hsCRP level of greater than 2 mg/L.

78. The method of claim 77, wherein the treatment is sufficient to reduce the hsCRP level to 2 mg/L or less.

79. The method of claim 78, wherein the treatment is sufficient to reduce the hsCRP level to 1 mg/L or less.

80. The method of claim 77, wherein the treatment is sufficient to reduce the hsCRP level by at least 50%, 60%, 70%, 80%, or 90% as compared to the pre-treatment level.

81. The method of claim 77, wherein the treatment results in hsCRP reduction after about 180 days of treatment.

82. The method of claim 70, wherein the patient has elevated serum or urinary hepcidin.

83. The method of claim 70, wherein the patient has a red blood cell distribution width (RDW) of greater than 13% or RDW in the highest quartile.

84. The method of claim 70, wherein the patient has a white blood cell count (WBC) of more than 9000 cells per microliter of blood or WBC in the highest quartile.

85. The method of claim 65, wherein the patient is negative for active tuberculosis, HIV and/or hepatitis B or C.

86. The method of claim 65, wherein the patient is at least 18 years old.

87. The method of claim 65, wherein at least one additional therapeutic agent is administered to the patient.

88. The method of claim 87, wherein the additional therapeutic agent is a therapeutically affective dose of an anti-diabetic drug.

89. The method of claim 65, wherein the dose is administered every one week to every 24 weeks.

90. The method of claim 87, wherein the dose is administered every 4 weeks, every 8 weeks, every 12 weeks or every 24 weeks.

91. The method of claim 65, wherein the dose is administered every 30 days to every 90 days.

92. The method of claim 89, wherein the dose is administered every 30 days or every 90 days.
